(19) United States
(12) Patent Application Publication
(10) Pub. No.: US 2009/0042951 A1
(43) Pub. Date: Feb. 12, 2009

(54) Blood Pressure Reduction in Salt-Sensitive Hypertension

(76) Inventor: Robert Danziger, Chicago, IL (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/588,673

(22) PCT Filed: Feb. 22, 2005
(86) PCT No.: PCT/US2005/005626

§ 371 Date: Sep. 17, 2007

Related U.S. Application Data
(60) Provisional application No. 60/546,227, filed on Feb. 20, 2004.
(60) Provisional application No. 60/549,289, filed on Mar. 2, 2004.

(51) Int. Cl. A61K 31/4015; A61P 9/12; A61K 31/44

(52) U.S. Cl. 514/352; 514/424

(57) ABSTRACT

This invention provides methods and compositions, most preferably pharmaceutical compositions, for treating salt-sensitive hypertension through the inhibition of certain enzymes in the beta-adrenergic pathway that are involved in the regulation of the secretion of water and sodium. These enzymes are cyclic nucleotide phosphodiesterases (PDE) that selectively hydrolyze the second messenger cAMP and, therefore, down-regulate beta-adrenergic signaling. Specifically provided are methods and pharmaceutical compositions for treating salt-sensitive hypertension by inhibiting certain members of the PDE4 family of cyclic nucleotide phosphodiesterases, particularly members of the PDE4B and PDE4D sub-families and, more particularly, the PDE4B1 and PDE4D5 isotypes thereof.

### RELATED APPLICATIONS

This application is related to U.S. provisional patent application, Ser. No. 60/546,227, filed Feb. 20, 2004 and U.S. provisional patent application, Ser. No. 60/549,289, filed Mar. 2, 2004, the disclosures of each of which are explicitly incorporated herein.

### BACKGROUND OF THE INVENTION

1. Field of the Invention

This invention relates to methods for treating salt-sensitive hypertension in mammals. This invention particularly relates to methods for using inhibitors of certain isoforms and splice variants of cyclic nucleotide phosphodiesterases for treating salt-sensitive hypertension in mammals.

2. Description of the Related Art

Hypertension is the most common treatable risk factor for death and disability from heart disease, stroke, and kidney failure throughout the world. However, less than twenty percent of the estimated 50 million Americans and over 500 million people worldwide who suffer from this disease have their blood pressures adequately controlled. Because the underlying cause of the hypertension is unknown in the overwhelming majority of cases, this disorder cannot be cured, but rather requires lifelong treatment with lifestyle modification and numerous medications. Compliance with the blood pressure monitoring, lifestyle modifications and medication schedules required to control this disease is difficult for patients to maintain, resulting in poor control rates in all countries. In addition, some forms of this disease are refractory to presently available treatments.

Genetic background plays a role in hypertension, with shared genes accounting for between about 30 and 50% of individual variability in blood pressure (Luft et al., 1995, J. Hypertens 13: 1535-1538; Grim et al., 1995, Hypertension: Pathophysiology, Diagnosis and Management, (Laragh, J. H., ed.), Raven Press, New York; Kwitek-Black & Jacob, 1999, Hypertension Primer, Williams & Wilkins, Baltimore, pp. 222-223). The genetic component is reflected in high blood pressure aggregating in families in a “dose-dependent” manner: the stronger a person's family history of hypertension the greater the risk of developing the disease. Luft et al., 1995, Id. These observations have fueled research into possible hypertension candidate genes (Jeunemaitre et al., 1992, Cell 71: 169-180) as potential diagnostic and therapeutic targets. However, much (50 to 70%) of the remaining variability appears to be environmentally related, with stress and dietary sodium intake being two of the major contributors.

Dietary sodium intake is one of the most important environmental determinants of human hypertension. The epidemiologic, clinical, and experimental support for this is overwhelming. For example, the Intersalt Study (The INTERSALT Study: background, methods, findings, and implications, 1997, Am. J. Clin. Nutr. 65(2 Suppl):626S-642S), which was conducted over a 30 year period at 52 locations around the world, clearly demonstrated that the risk of developing hypertension was linearly and very tightly related to 24-hour urine sodium excretion, the best measure of dietary sodium intake. Numerous lines of evidence indicate that impaired control of salt and/or water retention is a key factor in almost all manifestations of this disease. Numerous clinical studies in humans and extensive research based upon the many polygenetic rat models of salt-dependent hypertension that have been created have resulted in a detailed description of hypertension from the standpoint of organ systems physiology, but the precise genetic and regulatory underpinnings of the disease remain an enigma.

What is known is that the few rare known forms of hypertension that exhibit Mendelian inheritance have all been shown to involve excessive renal retention of salt and water, leading to severe salt-dependent hypertension. Furthermore, reduced dietary sodium intake in combination with diuretic administration has proven to be among the most effective treatments for both Mendelian and non-Mendelian forms of primary hypertension and is currently recommended as first line therapy in all of the major practice guidelines around the world. However, it is also well known that within the general patient population, both normotensive and hypertensive persons show tremendous inter-individual variability in their blood pressure responses to dietary sodium loading and sodium restriction. This variability indicates although there is a strong genetic underpinning for at least some forms of the disease, other regulatory factors are involved and may predominate in many cases.

As a consequence of the foregoing and with few exceptions, the current approach to clinical treatment of hypertension is almost entirely an empirical one. Generally accepted practice is to treat most forms of hypertension via dietary modifications that reduce salt intake in combination with the administration of diuretics, beta-adrenergic receptor blockers, aldosterone antagonists and inhibitors of renin-angiotensin-aldosterone (RAS) signaling, e.g., angiotensin converting enzyme (ACE) inhibitors, and angiotensin receptor blockers (ARBs).

Although these therapies are effective for many individuals, they are only partially effective or even ineffective for others. The availability of a new class of anti-hypertensive therapeutic agents that target metabolic and regulatory pathways other than those targeted by existing therapeutic agents would provide clinicians with additional options in finding treatments that are effective for individual patients. Furthermore, it is desirable to identify additional indications for the use of existing therapeutic agents in order to extend their commercial life and utility.

### SUMMARY OF THE INVENTION

The present invention provides methods for treating salt-sensitive hypertension through the inhibition of certain enzymes in the beta-adrenergic pathway that are involved in the regulation of water and sodium secretion. These enzymes are cyclic nucleotide phosphodiesterases (PDE) that selectively hydrolyze the second messenger cAMP and, therefore, down regulate beta-adrenergic signaling. Specifically, the methods of the invention inhibit certain members of the PDE4 family of cyclic nucleotide phosphodiesterases, particularly members of the PDE4B and PDE4D sub-families and, more particularly, the PDE4B1 and PDE4D5 isotypes thereof, and said inhibition is effective in reducing elevated blood pressure levels associated with salt-sensitive hypertension. Furthermore, the invention provides methods for inhibiting PDE4 cyclic nucleotide phosphodiesterases that are presently in clinical use for the treatment of indications other than hypertension, which inhibitors exhibit significant anti-hypertensive activity.

The invention also provides pharmaceutical compositions for treating salt-sensitive hypertension, comprising a therapeutically-effective amount of a PDE inhibitor and a pharmaceutically-acceptable carrier, diluent or adjuvant. In certain embodiments, the PDE inhibitor is Rolipram; 4-substituted-2-pyrrolidinones; N-substituted cis-tetra-hydrophthalazinones; N-substituted cis-hexa-hydrophthalazinones; substituted aminopyridines; L-791943; TVX2706; RP73401 or RS25344. As provided herein, the pharmaceutical composition inhibits PDE4 isoforms PDE4B or PDE4D or both PDE4B and PDE4D, more particularly the isoforms PE4B1 or PDE4D5 or both PDE4B1 and PDE4D5.

A particular example of such anti-hypertensive PDE4 inhibitors is rolipram, a drug conventionally used for treating depression, improving cognitive function and treating inflammatory conditions.

Specific preferred embodiments of the present invention will become evident from the following more detailed description of certain preferred embodiments and the claims.

### DESCRIPTION OF THE DRAWINGS

An understanding of the invention is facilitated by reference to the drawings.

FIGS. 1A and 1B show the effect of rolipram on blood pressure in salt-sensitive and salt-resistant rats.

FIG. 1A (Top) is a representation of six-day continuous telemetric blood pressure monitoring from a single Dahl salt-sensitive rat (Dahl SS/jr) fed a 8% NaCl diet. A single intraperitoneal injection of Rolipram (10 mg/kg) is administered at day 2 (arrow). FIG. 1A (Bottom) is a bar chart summarizing the blood pressure monitoring data from three Dahl salt-sensitive rat (Dahl SS/jr) fed a 8% NaCl diet. A single intraperitoneal injection of Rolipram (10 mg/kg) is administered at day 2.

FIG. 1B (Top) is a representation of six-day continuous telemetric blood pressure monitoring from a spontaneously hypertensive rat (SHR) fed an 8% NaCl diet. A single intraperitoneal injection of Rolipram (10 mg/kg) is administered at day 2 (arrow). FIG. 1B (Bottom) is a bar chart summarizing the blood pressure monitoring data from three salt-resistant rats (SHR) fed a 8% NaCl diet. A single intraperitoneal injection of Rolipram (10 mg/kg) is administered at day 2.

FIGS. 2A and 2B illustrate PDE4B isotype expression in the kidneys of Dahl salt-sensitive (SS) and salt-resistant (SR) rats fed low and high salt diets.

FIG. 2A is a Western Blot of kidney extracts from Dahl salt-sensitive (SS) and salt-resistant (SR) rats fed high and low salt diets. This gel was stained using a polyclonal rabbit primary antibody that reacts with all known PDE4B splice variants.

FIG. 2B is a bar graph summarizing the abundance of PDE4B1 determined in Dahl salt-sensitive (SS) and salt-resistant (SR) rat kidneys by Western Blot analysis (n=3; P<0.01).

FIGS. 3A and 3B illustrate expression of PDE4D isotypes in the kidneys of Dahl salt-sensitive (SS) and salt-resistant (SR) rats fed low and high salt diets.

FIG. 3A is a Western Blot of kidney extracts from Dahl salt-sensitive (SS) and salt-resistant (SR) rats fed high and low salt diets. This gel was stained using a polyclonal rabbit primary antibody that reacts with all known PDE4D splice variants.

FIG. 3B is a bar graph summarizing the abundance of PDE4D5 determined in Dahl salt-sensitive (SS) and salt-resistant (SR) rat kidneys by Western Blot analysis (n=3; P<0.05).

FIG. 4 illustrates rat kidney tissue slices that have been immunohistochemically stained for PDE4B1 (Top) and PDE4D5 (Bottom). The left-hand panel in each pair is from a Dahl SS/jr salt-sensitive rat while the right-hand panel is from a Dahl SR/jr salt-resistant rat. The “P” and “D” markers identify the proximal and distal tubules, respectively.

### DETAILED DESCRIPTION OF THE INVENTION

It is now generally accepted that many of the most pervasive illnesses including hypertension are rarely inherited in a Mendelian fashion; rather, they are polygenic in etiology, and gene-gene and gene-environment interactions are significant factors in the manifestation(s), prognosis and treatment of the disease state. As a result, considerable effort is being devoted to identifying the genes that contribute to and/or modify the behaviors of complex inherited diseases with the intent of developing both an understanding of the pathophysiological aspects of these diseases and of discovering novel drug targets.

Both functional and positional approaches have been utilized in attempts to identify the genes and suites of genes that are involved in the etiology and progression of various complex diseases. Functional approaches relying on the identification of candidate genes are based on a series of ‘educated’ guesses without reference to chromosomal map position. The major shortcomings of these methods are the requirement for a priori knowledge of gene function and the expectation that critical genes have a detectable impact on the phenotype. Conversely, positional approaches locate candidate genes solely on the basis of map position and obviate the need for a priori knowledge of gene function. However, the identification of the culprit gene or genes within the relevant region is a major challenge. Recently, the “positional candidate” method (Collins, 1995, Nat. Genet. 9: 347-350) has been used; this method is based on the combination of the identification of a chromosomal sub-region or quantitative trait locus (QTL), usually by linkage analysis, with an examination of the region to identify attractive candidate genes of known or suspected function. The weakness of this method is that it relies on the density of the transcript map and on a priori knowledge of gene function to identify good candidate genes within the region of interest. Moreover, the structural genetic approach does not address the biological path(s) that lead(s) from a normal allelic variation to vulnerability to an illness.

The above strategies, which focus on discovering genetic differences, have had limited success in identifying genes that could be robustly and verifiably associated with complex diseases. For this reason, recent approaches have been developed to globally assess gene function, including transcriptional profiling and determining alterations in protein levels. These methods, which are still in their early phases of development, offer the significant advantage of examining function specifically in the affected tissue, thereby defining a profile of altered activity that results from the interaction of genes and environment. By the same token, the observed alterations in gene function are, in the majority of cases, not likely to be primary or directly attributable to genetic variations. Moreover, many might also be secondary to the disease process rather than its cause. Thus, transcriptional profiling alone cannot define the genetic etiology of complex disorders.

The most recently developed methods (reviewed in Danziger et al., 2005, Current Hypertension Reviews 1: 21-34) combine global functional screening methods, such as transcriptional profiling, with genomic mapping and functional positional cloning techniques to provide a more complete picture of the genes and gene interactions involved in a complex disease. This is a demanding process requiring both genetic expertise and specific knowledge of the organ or physiological system involved in the disorder. Its utility further depends either on the availability of appropriate human tissue or an informative animal model of the disease as well as upon the power of the individual technologies and the ability to analyze, mine and interpret the resulting information.

The physiological control of blood pressure and corresponding disease states such as hypertension has a complex quantitative phenotype. The greatest insights into the genetics of hypertension have been developed with respect to the Mendelian forms (Carluccio et al., 2001, Eur. J. Clin Invest 31: 476-488) of the disease. Thus far, the identification of candidate genes has been most successful for genes for which there is a priori knowledge of relevance to the pharmacology and physiology of blood-pressure control, e.g., those in the renin-angiotensin-aldosterone (RAS) and adrenergic signaling pathways (Hansson et al., 1995, Nat Genet 11: 76-82).

Although dietary salt is considered to be a major environmental risk factor for hypertension, there is significant inter-individual variability in the blood-pressure response to salt intake, which indicates that hypertension has polygenic underpinnings. So far, a few genes (including aldosterone synthase and 11-beta hydroxylase, the epithelial sodium channel (ENaC), the mineralocorticoid receptor and the serine-threonine kinases, WNK1 and WNK4) have been linked to rare Mendelian forms of human hypertension that show some salt sensitivity (see Stewart et al, 1996, Lancet 347: 88-91; Mune et al., 1995, Nat. Genet. 10: 394-399; Busjahn et al., 2002, Hypertension 40: 256-260 (2002); Wilson et al., 2001, Science 293: 1107-1112; Lifton et al., 1992, Trans. Assoc. Am. Physicians 105: 64-71; Lifton et al., 1992, Nature 355: 262-265; Cover et al., 1995, J Biol. Chem 270: 16555-16560; Churchill et al., 1992, Am. J Physiol 262: H1809-H1817). However, the candidate genes that have been tested for salt-sensitivity and evaluated to date do not fully explain salt-sensitive hypertension. The need to identify novel genes that have not been considered and to highlight specific genes within regions that have been identified by physical-mapping strategies led to the use of the integrative approach of transcriptional profiling in conjunction with QTL analysis in animal models.

Studies of salt-sensitive hypertension have capitalized on several artificially selected, inbred hypertensive strains of rats, in which congenic constructs and genome-wide scans have been used to identify QTLs for blood pressure. One of the most commonly studied strains is the Dahl salt-sensitive (SS) rat strain, which is an inbred strain that was artificially selected from progenitor Sprague Dawley rats to obtain the maximum blood-pressure response when given a high-salt diet (Churchill et al., 1992, Am. J Physiol 262:H1809-H1817) This is considered as a “renal model” of salt-sensitive hypertension, as cross-transplantation experiments have shown that the kidney confers salt sensitivity (Dahl et al., 1967, J Exp. Med 126: 687-699). The utility of the Dahl rat in these studies is enhanced by the availability of a corresponding salt-resistant (SR) strain that does not exhibit a significant blood pressure response when fed a high-salt diet.

Congenic strains and genome-wide scans of the Dahl rat have identified up to 16 blood-pressure QTLs (Garrett et al., 2002, J. Hypertens. 20: 2399-2406; Garrett et al., 2000, Physiol Genomics 3: 33-38; Garrett et al., 1998, Genome Res 8: 711-723). A major challenge has been to identify the genes within these QTLs that are associated with salt-sensitivity. Genes for which the renal transcript abundance changes during normal adaptation to a high-salt diet will include those that are important for salt adaptation, those that are induced by hypertension, and those that are unrelated to salt-adaptation and blood pressure control. For this reason, attention was paid to those genes and gene transcripts whose expression changes differentially in response to changing levels of dietary salt intake in salt-sensitive (SS) and salt-resistant (SR) Dahl rats.

Three of the genes thus identified are elements of the renin-angiotensin-aldosterone (RAS) signaling pathway. This result was expected as several major classes of therapeutic agents in widespread clinical use for the treatment of hypertension specifically target elements of the RAS pathway. These classes of agents include angiotensin converting enzyme (ACE) inhibitors; angiotensin receptor blockers; and aldosterone antagonists as well as methyl-DOPA, a CNS agent that may modulate RAS signaling.

An unexpected result of this study was the discovery that certain members of a group of genes associated with the beta-adrenergic signaling pathway were differentially expressed by salt-sensitive and salt-resistant Dahl rats fed high salt diets. These genes were determined to encode cyclic nucleotide phosphodiesterase (PDE) enzymes that modulate adrenergic signaling through hydrolysis of intracellular “second messenger” molecules cAMP and cGMP. Subsequent experimentation revealed that administration of PDE inhibitors to hypertensive Dahl SS/Jr rats resulted in an unanticipated immediate and prolonged reduction in blood pressure. Further experimentation determined that the effect was observed only when certain members of a particular subset of PDEs known in the art that hydrolyze cAMP were inhibited.

The beta-adrenergic signaling pathway is believed to be the primary source of cellular cAMP. Beta-adrenergic agonists, acting through G-protein mediated coupling, stimulate adenylate cyclase, which converts ADP to cAMP. cAMP, in turn, stimulates specific cAMP-dependent protein kinases (PkAs) that phosphorylate a variety of proteins that directly or indirectly modulate cellular and physiological functions. Specific cyclic nucleotide phosphodiesterases (PDE) hydrolize cAMP to adenosine monophosphate and can, therefore attenuate or abrogate the effects of stimulated cAMP production. Thus, PDEs are in central position to regulate cAMP-mediated signaling, but the specific roles that PDEs play in normal cellular and physiological processes are largely unknown.

It was known that cAMP mediates cellular responses to a wide variety of hormones and neurotransmitters. As a consequence, processes that affect the cellular concentration of cAMP can, in turn, modulate metabolic and physiological processes as diverse as inflammation, platelet aggregation, secretion, cardiac and smooth muscle contraction, apoptosis, glycogenolysis, ion channel conductance and cell growth. This broad spectrum of activities has led to the development of numerous pharmacological agents including PDE inhibitors that exert therapeutic effects through alteration of intracellular cAMP concentrations. These agents have found clinical use as anti-inflammatories, anti-depressants, anti-asthmatics, smooth muscle relaxants, anti-thrombotics, vasodialators, cardiotonics, and agents for improving cognitive function. Prior to the present invention, however, these none of these agents had been identified as exhibiting anti-hypertensive properties.

Over sixty PDE isoforms encoded by 21 genes are presently known in humans. These PDE isoforms are categorized into eleven families based primarily upon substrate specificities. Members of PDE families 1, 2, 3, 10 and 11 hydrolyze both cAMP and cGMP, but each member of these families exhibit different Kₘ values for the two substrates. Members of the PDE-4, -7 and -8 families preferentially hydrolyze cAMP while members of the PDE-5, -6 and -9 families are cGMP specific. PDE4 in particular has both a high specificity and a high affinity for cAMP. All known PDEs appear to share a common three-domain structure consisting of a C-terminal catalytic domain that is largely conserved within each family; a N-terminal domain of variable structure that may be involved in the cellular and/or tissue localization of PDEs, and a central domain that contains regulatory structural motifs that are somewhat conserved within each family. Most PDE families consist of multiple isotypes that appear to be N-terminal splice or start variants of the family archetype. There is some evidence that the expression of these splice and start variants may be inducible by or modulated by changing physiological conditions.

Of the several classes of therapeutic agents that are in clinical use for the treatment of hypertension, (ACE inhibitors; angiotensin receptor blockers; aldosterone antagonists; and methyl-DOPA), none are known to be PDE agonists or inhibitors. Although there is evidence that the increased levels of cAMP resulting from the administration of rolipram, a known PDE4 inhibitor, to dogs both reduces renal vascular resistance and promotes secretion of electrolytes (Tanahashi, 1999, JPET 289:1533-1538), the roles that renal cAMP and renal PDEs play in physiological salt-adaptation and salt-sensitive hypertension were not known prior to this invention. Furthermore, the importance of certain PDE isoforms in these physiological processes and the use of PDE inhibitors in the treatment of salt-sensitive hypertension had not previously been demonstrated. However, recently the art has recognized the possibilities for a use for PDE4 inhibitors for treating pulmonary hypertension incidental to inflammation (see, for example, U.S. Patent Applications, Publication Nos. US 2005/0020587; US 2005/0020611; US 2005/0020626; US 2005/0020639, each of which is incorporated by reference). However, none of these references demonstrated this use.

Demonstration that PDE inhibitors in general and inhibitors of PDE4 in particular can exert anti-hypertensive effects was performed as described in the Examples below.

The invention provides pharmaceutical compositions comprising at 10 therapeutically effective amount of a PDE inhibitor. As used herein, an “effective amount” or “therapeutically effective amount” of a PDE inhibitor is defined as an amount that when administered to an animal, preferably a human, having salt-sensitive hypertension, reduces the blood pressure in the animal. The “effective amounts” of said PDE inhibitors are those doses that produce subnanomolar to millimolar concentrations of a compound such as rolipram in blood or plasma, and will depend on species, pharmacokinetics, and route of administration.

Pharmaceutical compositions of the PDE inhibitors of the present invention can be formulated and administered through a variety of means, including systemic, localized, or topical administration. Techniques for formulation and administration can be found in “Remington's Pharmaceutical Sciences,” Mack Publishing Co., Easton, Pa. The mode of administration can be selected to maximize delivery to a desired target site in the body. Suitable routes of administration can, for example, include oral, rectal, transmucosal, transcutaneous, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one can administer the PDE inhibitors of the present invention in a local rather than systemic manner, for example, via injection of the compound directly into a specific tissue, often in a depot or sustained release formulation.

Pharmaceutical compositions for use in accordance with the methods of the present invention thus can be formulated in conventional manner using one or more physiologically acceptable carriers comprising excipients and auxiliaries that facilitate processing of PDE inhibitors into preparations that can be used pharmaceutically. Proper formulation is dependent upon the route of administration chosen.

The compounds of the present invention can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the PDE inhibitors in water-soluble form. Additionally, suspensions of the compounds of the present invention can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances that increase the viscosity of the suspension, such as sodium carboxymethyl cellulose, sorbitol, or dextran. Optionally, the suspension can also contain suitable stabilizers or agents that increase the solubility of the compounds to allow for the preparation of highly concentrated solutions. Alternatively, the active ingredient can be in powder form for constitution with a suitable vehicle, e.g., sterile pyrogen-free water, before use. The compounds can also be formulated in rectal compositions such as suppositories or retention enemas, e.g. containing conventional suppository bases such as cocoa butter or other glycerides.

For injection, compounds of the present invention can be formulated in appropriate aqueous solutions, such as physiologically compatible buffers such as Hank's solution, Ringer's solution, lactated Ringer's solution, or physiological saline buffer. For transmucosal and transcutaneous administration, penetrants appropriate to the barrier to be permeated are used in the formulation. Such penetrants are generally known in the art.

For oral administration, PDE inhibitors of the present invention can be formulated readily by combining the PDE inhibitors with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate. Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the PDE inhibitors in admixture with filler such as lactose, binders such as starches, and/or lubricants such as talc or magnesium stearate and, optionally, stabilizers. In soft capsules, PDE inhibitors can be dissolved or suspended in suitable liquids, such as fatty oils, liquid paraffin, or liquid polyethylene glycols. In addition, stabilizers can be added. All formulations for oral administration should be in dosages suitable for such administration. For buccal administration, the compositions can take the form of tablets or lozenges formulated in conventional manner.

For administration by inhalation compounds of the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

In addition to the formulations described previously PDE inhibitors of the present invention can also be formulated as a depot preparation. Such long acting formulations can be administered by implantation (for example subcutaneously or intramuscularly) or by intramuscular injection. Thus, for example, the PDE inhibitors can be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for hydrophobic embodiments of the PDE inhibitors of the present invention is a co-solvent system comprising benzyl alcohol, a nonpolar surfactant, a water-miscible organic polymer, and an aqueous phase. The co-solvent system can be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system can be varied considerably without destroying its solubility and toxicity characteristics. Furthermore, the identity of the co-solvent components can be varied: for example, other low-toxicity nonpolar surfactants can be used instead of polysorbate 80; the fraction size of polyethylene glycol can be varied; other biocompatible polymers can replace polyethylene glycol, e.g. polyvinyl pyrrolidone; and other sugars or polysaccharides can substitute for dextrose.

Alternatively, other delivery systems can be employed. Liposomes and emulsions are well known examples of delivery vehicles or carriers for hydrophobic drugs. Certain organic solvents such as dimethylsulfoxide also can be employed, although usually at the cost of greater toxicity. Additionally, PDE inhibitors of the present invention can be delivered using a sustained-release system, such as semipermeable matrices of solid hydrophobic polymers containing the therapeutic agent. Various sustained-release materials have been established and are well known by those skilled in the art. Sustained-release capsules can, depending on their chemical nature, release the PDE inhibitors of the present invention for a few weeks up to over 100 days.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Pharmaceutical compositions suitable for use in the present invention include compositions wherein the PDE inhibitors are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

The invention also provides formulations of the PDE inhibitors of the present invention which as foodstuffs, food supplements or as a component of a food for an animal, preferably a human, more preferably a human with salt-sensitive hypertension.

For any PDE inhibitors of the present invention used in the method of the invention, the therapeutically effective dose can be estimated initially from in vitro assays, as disclosed herein, or using art-recognized animal model systems or a combination thereof. For example, a dose can be formulated in animal models to achieve a circulating concentration range that includes the EC₅₀ (effective dose for 50% increase) as determined in vitro, i.e., the concentration of the test compound which achieves a half-maximal amount of reduction in hypertension. Such information can be used to more accurately determine useful doses in humans.

It will be understood, however, that the specific dose level for any particular patient will depend upon a variety of factors including the activity of the PDE inhibitors employed, body weight, general health, sex, diet, time of administration, route of administration, and rate of excretion, drug combination, the severity and extent of the particular salt-sensitive hypertension in the patient undergoing therapy and the judgment of the prescribing physician and in particular the age of the patient.

Preferred PDE inhibitors of the present invention will have certain pharmacological properties. Such properties include, but are not limited to oral bioavailability, low toxicity, low serum protein binding and desirable in vitro and in vivo half-lives. Assays may be used to predict these desirable pharmacological properties. Assays used to predict bioavailability include transport across human intestinal cell monolayers, including Caco-2 cell monolayers. Serum protein binding may be predicted from albumin binding assays. Such assays are described in a review by Oravcová et al. (1996, J. Chromat. B 677: 1-27). In vitro half-lives of PDE inhibitors of the present invention may be predicted from assays of microsomal half-life as described by Kuhnz and Gieschen (1998, Drug Metabolism and Disposition, 26: 1120-1127).

Toxicity and therapeutic efficacy of the compounds of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₆₀ and ED₅₀. PDE inhibitors of the present invention that exhibit high therapeutic indices are preferred. The data obtained from these cell culture assays and animal studies can be used in formulating a range of dosage for use in humans. The dosage of such PDE inhibitors of the present invention lies preferably within a range of circulating concentrations that include the ED₅₀ with little or no toxicity. The dosage can vary within this range depending upon the dosage form employed and the route of administration utilized. The exact formulation, route of administration and dosage can be chosen by the individual physician in view of the patient's condition. (See, e.g. Fingl et al., 1975, in “The Pharmacological Basis of Therapeutics”, Ch. 1, p.1).

Dosage amount and interval of administration of PDE inhibitors of the present invention can be adjusted individually to reduce seizure frequency, duration or intensity. For particular embodiments of the PDE inhibitors of the present invention, dosage amount and timing of administration of said inhibitors can be adjusted individually to provide plasma levels of the inhibitors that are sufficient to reduce salt-sensitive hypertension in the animal.

### EXAMPLES

The Examples which follow are illustrative of specific embodiments of the invention, and various uses thereof. They set forth for explanatory purposes only, and are not to be taken as limiting the invention.

### Example 1

### Effect of PDE4 Inhibitors on Blood Pressure

The effect of PDE4 inhibitors on salt-sensitive hypertension was demonstrated using male Dahl salt-sensitive (SS/Jr) rats (250-300 gm, Harlan, Indianapolis, Ind.) that were placed on basal (0.3%) or high (8%) NaCl rat chow diets (Purina series 5500) beginning ten days prior to the start of the experiments. Male Dahl salt-resistant (SR/Jr) rats (250-300 gm, Harlan, Indianapolis, Ind.) were used as controls. Non-salt-sensitive, spontaneously hypertensive rats (SHR) were also employed in some experiments. Each rat was housed individually with a 12 hour light/12 hour dark cycle. The animal use protocols were approved by the Chicago—West Side Veterans Administration Institutional Animal Care and Use Committee.

Arterial blood pressures in unrestrained rats were monitored using a model TA11PA-C20 (Data Science, International, St. Paul, Minn.) implantable telemetric blood pressure monitoring system. The rats were anesthetized using an intraperitoneal injection (50 mg/kg i.p.) of nembutol sodium solution prior to implantation of the monitor. The radio-transmitter catheter was inserted via a midline abdominal incision into the left femoral artery and sutured in place after the tip of the catheter was advanced into the proximal aorta. The catheter was tunneled under the skin and the body of the transmitter was positioned in a subcutaneous pocket near the right flank of the rat. Data acquisition began after normal diurnal blood pressure variability had been reestablished, typically about seven days after surgery. Continuous measurements of pulsatile central aortic blood pressure were recorded by a receiver placed under each rat's cage.

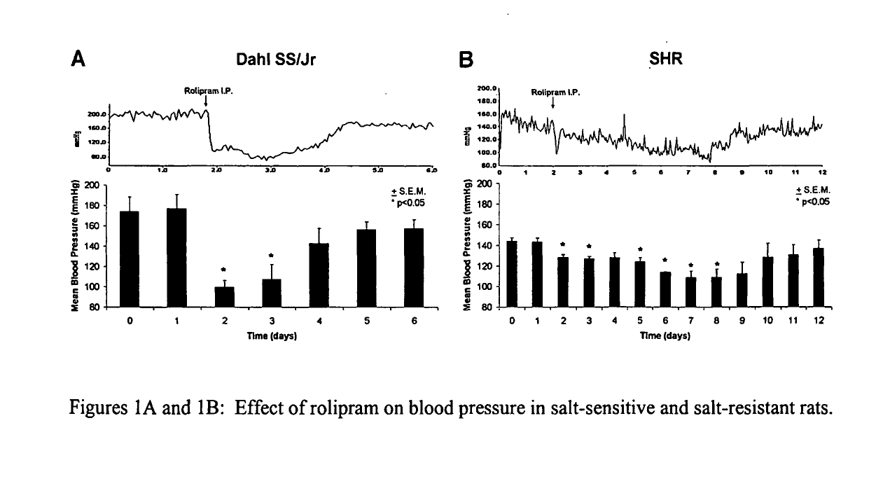

A baseline arterial blood pressure was established for each rat from continuous measurements made over a two day period prior to the start of an experiment. Rolipram was injected intraperitoneally (IP) at 10 mg drug per kg of body weight, while a bolus of another PDE4 inhibitor was given at 2 mg drug per kg body weight. Monitoring continued for four additional days. FIGS. 1A and 1B illustrate the effects of rolipram injection on the arterial blood pressure of salt-sensitive (FIG. 1A) and salt-resistant (FIG. 1B) rats fed an 8% salt diet. FIG. 1A (Top) shows a typical 6 day continuous arterial blood pressure recording made from a Dahl SS/Jr rat in which a very significant and prolonged (>2 days) drop in blood pressure in response to rolipram injection is clearly evident. FIG. 1B (Top) shows the corresponding 12 day record from a spontaneously hypertensive (SHR) rat under these same conditions. This recording shows a brief, shallow dip in blood pressure immediately after rolipram injection followed by a gradual decline and recovery of blood pressure that is of significantly less magnitude and of longer duration than is observed in the salt-sensitive rats of FIG. 1A (Top). FIGS. 1A (Bottom) and 1B (Bottom) are bar charts summarizing these data for three rats in each group. The p-value in both cases is less than 0.05. As rolipram is known to be a selective inhibitor of PDE4, these results suggest that PDE4 activity is linked to salt-sensitive hypertension. The difference in the kinetics of the response to rolipram injection suggests that different mechanisms are acting in salt-sensitive and salt-resistant rats.

Similar results were obtained by oral administration of PDE4 inhibitor L-000826141, dosed at 1 mg/kg once daily in 0.5 or 1% aqueous methylcellulose solution, which reduced blood pressure in Dahl SS rats on a high salt diet.

### Example 2

### Expression of PDE4 Isotypes

Male Dahl salt-sensitive (SS/Jr) and salt-resistant (SR/Jr) rats (250-300 gm, Harlan, Indianapolis, Ind.) that were fed basal (0.3%) and high (8%) NaCl rat chow diets (Purina series 5500) for ten days were sacrificed and their kidneys excised. The kidneys were washed with phosphate-buffered saline (PBS, pH 7.4) and homogenized in a lysis buffer (20 mmol/L Tris-HCl, 150 mmol/L Na₂EDTA, 1 mmol/L EGTA 1% Triton 2.5 mmol/L sodium pyrophosphate, 1 mmol/L b-glycerolphosphate-1 mmol/L Na₃VO₄ ) supplemented with 1 mmol/L phenylmethanesulfonylfluoride (PMSF) and 1× protease inhibitors (Sigma-Aldrich, St. Louis, Mo.). The homogenate was spun at 14 000 rpm for 30 minutes at 4° C., and the supernatant was collected and stored in aliquots at −80° C. until use. Protein concentrations were estimated on each lysate with Bradford's reagent (Bio-Rad Laboratories, Hercules, Calif.). Samples were separated on a 10% SDS-polyacrylamide gel and the protein bands transferred to nitrocellulose membranes using Mini-PROTEAN® 3 Cell Transfer System (Bio-Rad Laboratories, Hercules, Calif.).

The membranes were immunoprobed with rabbit polyclonal primary antibodies specific for PDE4 and its isotypes (Fabgennix Inc. International, Shreveport, La.) in TBST (150 mM NaCl, 2 mM KCl, 25 mM Tris-Cl, 0.05% Tween 20) with 5% nonfat milk solution; treated with goat anti-rabbit IgG HRP-conjugated secondary antibody (Abcam Inc., Cambridge, Mass.); and the blot developed using the ChemiLuminescent Western Blot Detection kit (Pierce, Rockford, Ill.). Membranes were then stripped and immunoprobed with □-actin antibody. Densitometric analysis of the Western blots was carried out by quantifying the band density using ImageJ 1.30 v program (NIH, Research Service Branch, Bethesda, Md).

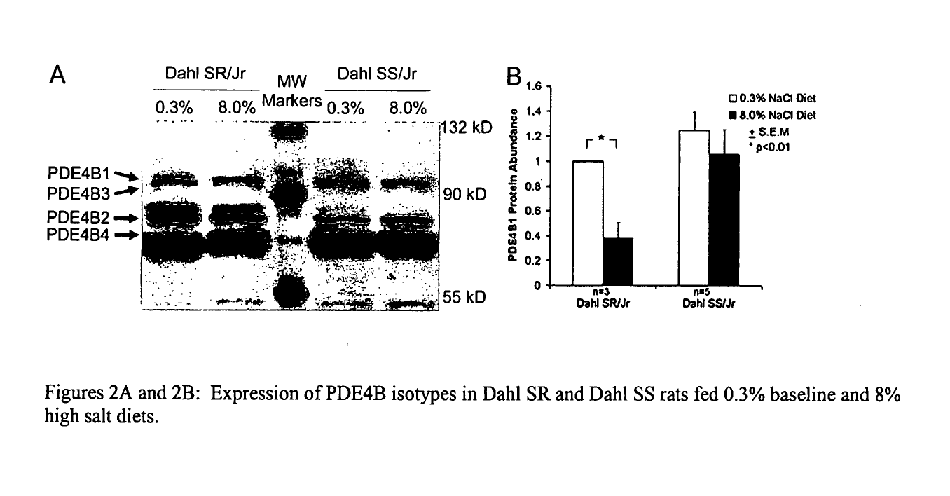

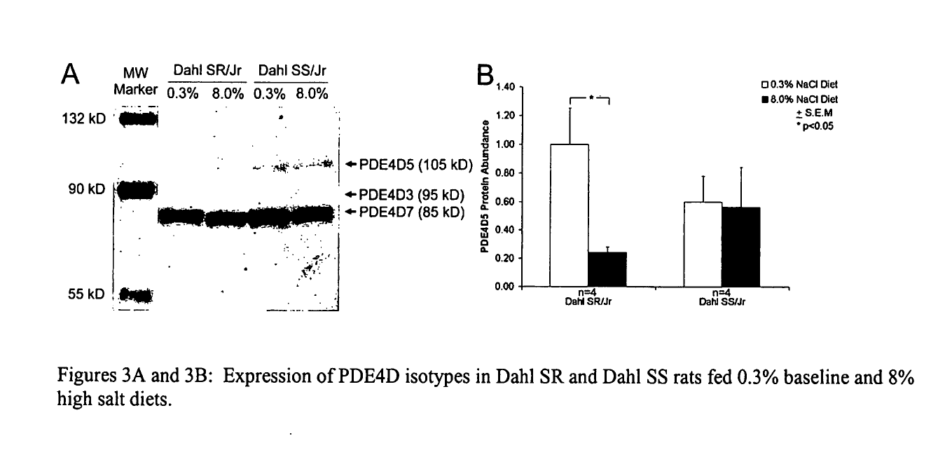

FIG. 2A shows a typical Western Blot that illustrates the differences in PDE4B isotype expression in the kidneys of salt-sensitive (SS/Jr) and salt-resistant (SR/Jr) rats fed basal (0.3%) and high (8%) NaCl rat chow diets. FIG. 2B is a bar chart that summarizes these data for the PDE4B1 isotype (n=3). It can be seen in FIGS. 2A and 2B that the expression of the PDE4B1 isoform is substantially reduced in the kidneys of salt-resistant rats fed a high salt diet whereas a much smaller and possibly not statistically significant change in PDE4B1 expression is observed in the kidneys of salt-sensitive rats. FIGS. 3 A and B show similar data for the expression of PDE4D5 in salt-resistant rats as a function of dietary salt intake.

These data and the corresponding data for the other renal PDE4 isotypes show that the expression of certain members of the PDE4B and PDE4D families including, particularly, PDE4B1 and PDE4D5, is significantly modulated by dietary salt intake in salt-resistant rats, but that this modulation is minimal in salt-sensitive rats under the same conditions. When rolipram or other PDE4 inhibitors are administered to hypertensive salt-sensitive rats on a high salt diet, the resulting inhibition of PDE4 reduces the rate of cAMP hydrolysis by these enzymes thereby allowing the concentration of cAMP to increase. This increased cAMP concentration stimulates the secretion of both salt and water in the urine which, through a sequence of physiological processes, is reflected in a decrease in blood pressure toward the levels observed in salt-resistant rats on the same diet. This is consistent with the known activity of rolipram as a selective inhibitor of PDE4B and PDE4D (IC₅₀ values of 130 nM and 240 nM, respectively).

The results obtained in the manner of Example 2 indicate that expression and activity of certain PDE4 isotypes, particularly PDE4B1 and PDE4D4, are involved in regulating salt tolerance in mammalian kidneys, and that over expression of these PDE4 isotypes can render the mammal salt-sensitive. These data in combination with the data from Example 1 indicate that this salt sensitivity manifests as hypertension in mammals fed a high salt diet and that this hypertension can be abrogated by treatment of the mammal with a selective inhibitor of PDE4 isotypes. Therefore, it can be concluded that selective PDE4 inhibitors can find utility in the treatment of salt-sensitive hypertension. Although PDE inhibitors have been extensively used clinically for a variety of indications, the utility of these agents in the treatment of salt-sensitive hypertension had not previously been identified.

Rolipram, a 4-substituted-2-pyrrolidinone, is a PDE4-selective inhibitor that is generally prescribed as an anti-depressant and for the improvement of cognitive function. Other PDE4-selective inhibitors are employed as anti-inflammatory drugs for the treatment of asthma and COPD. In addition to rolipram, other 4-substituted-2-pyrrolidinones; N-substituted cis-tetra-hydrophthalazinones; N-substituted cis-hexa-hydrophthalazinones; substituted aminopyridines; and the drugs L-791943; TVX2706; RP73401 and RS25344 have been found by the methods described herein to be PDE4 inhibitors that exhibit anti-hypertensive activity. Other PDE4 inhibitors such as those described in US patent applications 2005/0020587; US 2005/0020611; US 2005/0020626; US 2005/0020639 may likewise find use as anti-hypertensive agents.

### Example 3

### Immunobistochemical Localization of PDE4 Isotypes

PDE4 activity has been reported in inflammatory cells, gonads, cardiac muscle, liver, tracheal smooth muscle, various regions of the brain, and in kidney, but little information on the localization of PDE4 isotypes is available. Immunohistological methods were employed to verify the localization of PDE4, PDE4B, PDE4D, PDE4B1 and PDE4D5 activity in rat kidneys. A standard three-stage indirect immunoperoxidase technique was used for this purpose.

Briefly, tissue sections that had been fixed in 10% formaldehyde were rehydrated in graded alcohols (from 100% to 80%) and then rinsed in a running water bath. Endogenous peroxidase activity was quenched by preincubating the specimens in 3% hydrogen peroxide for 5 minutes at room temperature (RT). After washing in TBST, the specimens were incubated in serum-free protein blocking solution (DakoCytomation, Carpinteria, Calif.) for 30 minutes at RT and washed again in TBST. Three hundred μL of primary antibody (1:100 dilution) was applied, and the tissue was incubated for one hour in a humidity chamber at RT. Control tissues were processed similarly except that antibody diluent (DakoCytomation, Carpinteria, Calif.) was used in place of the primary antibody solution. After being washing again in TBST, the specimens were incubated with 300 μL of anti-rabbit HRP-abelled polymer (DakoCytomation, Carpinteria, Calif.) for 30 minutes at RT. Tissues were washed in TBST. Color development was performed by incubating the specimens with the liquid DAB substrate-chromogen system (DAKO) for 8 minutes in accordance with the manufacturers' directions. After final washes in TBST and distilled water, the slides were counterstained with hematoxylin for 2 minutes, dehydrated in graded alcohols, and mounted with a coverslip using Permount (Sigma-Aldrich, St. Louis, Mo.) according to standard methods.

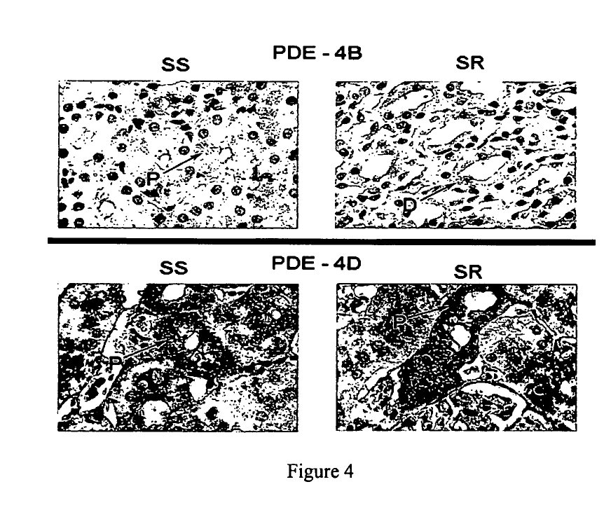

All specimens were subjected to microscopic analysis using a Nikon E600 (Melville, N.Y.) microscope with Axioplan objectives. Images of the stained specimens were captured using a Microlumina ultraresolution scanning digital camera [3,380×2,700 pixels (Leaf Systems, Fort Washington, Pa.)] and analyzed using Spot image analysis software (Diagnostic Instruments, Sterling Heights, Mich.). FIG. 4 shows representative images of renal tissue sections from salt-sensitive (left panes) and salt-resistant (right panes) rats that were immunohistochemically stained for PDE4B (top panels) and PDE4D (bottom panels). The arrows marked “P” and “D” indicate proximal and distal tubules, respectively. The PDE 4B is distributed in both proximal and distal tubules where as PDE4D is expressed in proximal tubules and undetectable in distal tubules.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

We claim:

1. A method of treating salt-sensitive hypertension in a mammal suffering therefrom, said method comprising the step of administering a therapeutically effective amount of a cyclic nucleotide phosphodiesterase (PDE) inhibitor to said mammal.

2. The method of claim 1 wherein the PDE is a PDE that preferentially hydrolyzes cAMP.

3. The method of claim 2 wherein the PDE is PDE4B or PDE4D or both PDE4B and PDE4D.

4. The method of claim 2 wherein the PDE is a renal isoform or splice variant of PDE4B and PDE4D.

5. The method of claim 2 wherein the PDE is PDE4B1 or PDE4D5 or both PDE4B1 and PDE4D5.

6. The method of claim 1 wherein the inhibitor is an inhibitor of PDEs that preferentially hydrolyze cAMP.

7. The method of claim 6 wherein the inhibitor is an inhibitor of PDE4B or PDE4D or both PDE4B and PDE4D.

8. The method of claim 6 wherein the inhibitor is an inhibitor of renal isoforms and splice variants of PDE4B and PDE4D.

9. The method of claim 2 wherein the inhibitor is an inhibitor of PDE4B1 or PDE4D5 or both PDE4B1 and PDE4D5.

10. The method of claim 6 wherein the PDE inhibitor is a 4-substituted-2-pyrrolidinone.

11. The method of claim 10 wherein the PDE inhibitor is Rolipram (ZK-62711).

12. The method of claim 6 wherein the PDE inhibitor is Rolipram; 4-substituted-2-pyrrolidinones; N-substituted cis-tetra-hydrophthalazinones; N-substituted cis-hexa-hydrophthalazinones; substituted aminopyridines; L-791943; TVX2706; RP73401 or RS25344.

13. The method of any one of claims 1-12 wherein the mammal is human.

14. A pharmaceutical composition for treating salt-sensitive hypertension, comprising a therapeutically-effective amount of a PDE inhibitor and a pharmaceutically-acceptable carrier, diluent or adjuvant.

15. A pharmaceutical composition according to claim 14, wherein the PDE inhibitor is Rolipram; 4-substituted-2-pyrrolidinones; N-substituted cis-tetra-hydrophthalazinones; N-substituted cis-hexa-hydrophthalazinones; substituted aminopyridines; L-791943; TVX2706; RP73401 or RS25344.

16. A pharmaceutical composition according to claim 14 wherein the PDE inhibitor inhibits PDE4B or PDE4D or both PDE4B and PDE4D.

17. A pharmaceutical composition according to claim 16 wherein the PDE inhibitor inhibits PDE4B1 or PDE4D5 or both PDE4B1 and PDE4D5.
